(19) United States
(12) Patent Application Publication
(10) Pub. No.: US 2022/0370193 A1
(43) Pub. Date: Nov. 24, 2022

(54) ENDOPROSTHESIS AND METHOD OF MANUFACTURING AN ENDOPROSTHESIS

(71) Applicant: KARDIOZIS SAS, Aix-en-Provence (FR)

(72) Inventors: Philippe POULETTY, Paris (FR); Daniel HAYOZ, Villars-sur-Glâne (CH); Nathalie VANDAELE-FENOUIL, Nanterre (FR)

(21) Appl. No.: 17/761,748

(22) PCT Filed: Sep. 19, 2019
(86) PCT No.: PCT/IB2019/000929

§ 371 Date: Mar. 18, 2022

(51) Int. Cl. A61F 2/07; A61F 2/00; B33Y 80/00

(52) U.S. Cl. CPC A61F 2/07; A61F 2/0077; B33Y 80/00; A61F 2250/0067; A61F 2250/001; A61F 2220/0075; A61F 2250/0071; A61F 2220/005; A61F 2240/002; A61F 2210/009; A61F 2210/0076; A61F 2002/077

(57) ABSTRACT

The invention relates to an endoprosthesis (1), in particular a vascular stent or a heart stent, comprising at least one body (3) part. At least one area (5,6) of an outer surface, preferably the whole outer surface, of the at least one body part (3) is provided with thrombogenic fibers (2). The invention further relates to methods of manufacturing endoprostheses (1).

The present invention relates to an endoprosthesis and a method for manufacturing an endoprosthesis according to the preamble of the independent claims.

Endoprostheses, in particular vascular and heart stents, are used to support blood vessels in the human body. For example, occlusions or aneurysms can be treated by placing such an endoprosthesis at the respective treatment site. In the treatment of an occlusion, the endoprosthesis keeps the vessel open for un-hindered blood flow. In the case of an aneurysm, the endoprosthesis can prevent circulation of blood in the aneurysm and thus lower the risk of a thrombus, rupture or further growth of the aneurysm.

It is known in the prior art to use thrombogenic elements on endoprostheses. For example, WO 2013/182614 A1 discloses an endoprosthesis with thrombogenic elements that extend away from a body of the endoprosthesis and promote thrombosis. This allows for the occlusion of an aneurysm to enhance the above-mentioned treatment effect.

However, currently known methods do not provide a simple way of post-production arrangement of thrombogenic elements on an endoprosthesis. Fixation and attachment of thrombogenic elements is usually cumbersome and difficult, and not typically versatile. In addition, they are limited to generic thrombus generation means that are not adapted to patient-specific needs.

Thus, the object of the present invention is to overcome the drawbacks of the prior art, in particular to provide an endoprosthesis and a method to produce an endoprosthesis wherein thrombogenic elements can easily be added to a surface of the endoprosthesis, in particular in a versatile manner and at selected locations on the endoprosthesis surface, and wherein the thrombogenic elements can be adapted to the patient-specific needs.

This and other objects are achieved by the endoprosthesis and the methods according to the characterizing portion of the independent claims of the invention.

The endoprosthesis, preferably the vascular stent or heart stent, according to the invention comprises at least one body part. At least one area of the outer surface of the at least one body part is provided with thrombogenic fibers. Preferably, the whole outer surface of the at least one body part is provided with thrombogenic fibers.

Thrombogenic fibers shall be understood as fibers that, in contact with blood, induce and/or promote to the formation of a thrombus. In particular, this process may involve the aggregation of platelets and red blood cells.

Preferably, the thrombogenic fibers are biodegradable and/or adapted to elute a drug. In particular, the fibers may be adapted to degrade in the human body within a certain time frame, for example one year, preferably six months, even more preferably three months. In addition, the material of which the fibers are made can be adapted to be resorbed by the human body. The ability of eluting a drug can be provided independently of the biodegradability, for example by coating the surface of the fibers with a drug than is taken up by the human body. However, it is also possible to combine both effects, for example by incorporating a drug in the fibers such that the degradation of the fibers releases the drug. This is particularly advantageous if the rate of drug elution is to be tuned to a desired level.

Preferably, at least one of the thrombogenic fibers comprises a biomarker or a biosensor. A biomarker enables the detection of a property or characteristic of the fiber, while a biosensor may enable the collection of additional data. For example, a biomarker can be used to detect whether a biodegradable fiber has already degraded, or to what extent. A biosensor could be used to measure characteristics such as inflammation levels, local temperature, or level of blood coagulation.

Preferably, the thrombogenic fibers are made of an elastic material and are adapted to expand upon deployment of the endoprosthesis. The elastic force can push the fibers away from the endoprosthesis body and thus enable a more reliable deployment of fibers.

Preferably, the surface of the thrombogenic fibers is adapted to increase the retention of a thrombus.

Preferably, the thrombogenic fibers comprise at least one of the group of micro-hooks, secondary fibers, loops, knots, or a texture. In particular, the retention of a thrombus is increased by these features. It is particularly advantageous if the surface of the fibers comprises said features. For example, the surface of the fibers can be textured, comprise micro-hooks, or loops. However, it also possible to adapt the fibers themselves as loops or with knots. Micro-hooks shall be understood as any elongated portion of a material that comprises a curved or intented portion to engage tissue or another material and that is substantially smaller than a fiber. In particular, a micro-hook may be pointed. Its size may be in the range of micrometers, but the term shall not be limited to that range.

Preferably, the length, diameter, and/or the density of the fibers are adapted to minimize or prevent endoleaks. For example, if the anatomy of the patient is such that endoleaks are expected at an end (e.g. a distal or proximal end) of the implant location, the density of fibers may be increased in that area.

Preferably, the length, diameter, and/or density of the thrombogenic fibers are optimized based on the characteristics of the aneurysm to be treated. For example, it may be advantageous to increase the number of fibers and their length around at a site that is intended to be located around a circumference of an aneurysm.

Preferably, the thrombogenic fibers comprise an active agent, in particular a protein and/or an enzyme that promotes clot formation and/or inhibits thrombolysis and/or proteolysis.

In particular, the active agent may inhibit at least one of plasmin and metallo protease.

Preferably, the thrombogenic fibers comprise a pharmaceutical substance, in particular an enzyme and/or a protein, that promotes and/or increases coagulation.

Preferably, the thrombogenic fibers comprise two ends, both of which are attached or attachable to the endoprosthesis by way of two separate anchor points.

Preferably, the shortest distance between the anchor points along the surface of the endoprosthesis in its deployed form is shorter than the length of the fiber that is connected to the two anchor points. This ensures that the fiber, in the deployed state, extends away from the surface of the endoprosthesis.

Preferably, at least one of the thrombogenic fibers extends around the longitudinal axis of the endoprosthesis at least once. In particular, it may be oriented along a circumference of the endoprosthesis. One extension around the longitudinal axis shall be understood as a full loop such that every hypothetical plane that is contiguous with the longitudinal axis of the endoprosthesis cuts the fiber at least twice.

Preferably, the endoprosthesis comprises a strip or a suture that extends substantially along the longitudinal axis. The strip or suture is attached or attachable to the at least one thrombogenic fiber at at least one attachment point. In particular, the strip or suture attaches, preferably permanently, the fiber or fibers to which it is attached to the endoprosthesis.

Preferably, at least one thrombogenic fiber that is oriented along a circumference is cuttable or cut at at least one cutting point. Upon cutting, the fiber comprises cut ends that extend away from the attachment point of the strip or suture.

Preferably, the thrombogenic fibers are attached to the endoprosthesis along their length by means of an adhesive composition. The adhesive composition may be biodegradable such that the fibers are released from the surface of the endoprosthesis occurs post-implantation due the degradation of the adhesive composition. In particular, the rate of biodegradation may be tunable, for example such that the fibers are completely released along their length after one year, or six months, or two months.

Preferably, the adhesive composition comprises at least one of the group of sugar, mannitol, or poly(lactid acid). It is of course possible to use any combination or concentration of these substances. It may also be advantageous to use different poly(lactic acid) with a particular polymerization degree, or a combination of different polymerization degrees, and/or a particular tacticity.

Preferably, the diameter of at least one of the thrombogenic fiber, is varying from one end of the fiber towards another end of the fiber over at least a section of the fiber. In particular, it may vary continuously from a proximal to a distal end. Even more preferably, all thrombogenic fibers have a diameter that varies in such a manner.

Preferably, the endoprosthesis comprises a fabric with directly integrated thrombogenic fibers.

In particular, the fabric may be woven, braided, or knitted.

Preferably, the thrombogenic fibers comprise a foot that promotes attachment into an endoprosthesis layer. The attachment may be magnetic, chemical, and or mechanical. In particular, a foot shall be understood as an end of a fiber comprising a means to attach to another site. It shall not be limiting to any particular shape or attachment mechanism.

Preferably, the endoprosthesis comprises at least two different types of thrombogenic fibers. This is particularly advantageous to adapt the endoprosthesis to specific patient needs. For example, the characteristics of an aneurysm to be treated in a particular patient may not be suitable for any one type of fiber.

Thus, the endoprosthesis can be adapted with two different types of fibers to account for the characteristics of the aneurysm in the patient. Of course, the same effect can be achieved with more than two types of fibers, or for other treatments than aneurysms.

Preferably, the at least two different types of thrombogenic fibers differ in at least one of length, diameter, or composition.

Preferably, the endoprosthesis comprises at least one nonthrombogenic fiber. In particular, the non-thrombogenic fiber may be one of a drug-eluting fiber, a fiber comprising a biomarker, and a fiber comprising a biosensor.

Preferably, at least a part of the endoprosthesis is manufactured by additive manufacturing.

Even more preferably, at least the fibers are manufactured by additive manufacturing.

In a particularly preferred embodiment, the whole endoprosthesis is manufactured by additive manufacturing.

Preferably, the thrombogenic fibers are provided with a connection interface for separately attaching the fiber to the endoprosthesis. This allows for a subsequent fixation of the fibers to the endoprosthesis. In particular, existing endoprosthesis may be retrofitted with connection interfaces so as to allow attachment of fibers.

In particular, the connection interface is adapted to establish a connection based on magnetic forces.

Preferably, the endoprosthesis comprises an inner and an outer layer wherein the thrombogenic fibers are attached or attachable to the outer layer, and the outer layer is attached or attachable to the inner layer.

In particular, the inner and the outer layer are attached or attachable by means of a glue and/or a suture.

Preferably, the color of the endoprosthesis can be selected from a variety of colors. In particular, a color code may be used to differentiate between different types of fibers. For example, the color of the endoprosthesis may be adapted such that the corresponding wavelength correlates to the length of the fibers on its surface. A violet endoprosthesis would then have the shortest fibers, and a red one the longest.

Preferably, the endoprosthesis comprises at least two pressure sensors, wherein one each is arranged on the inside and the outside of the endoprosthesis, respectively. This allows for measuring the pressure difference and/or decrease between an aneurysm and the blood vessel.

Preferably, the endoprosthesis is adapted to allow for the injection of thrombin in an aneurysm. For example, a catheter may be arranged around a graft wall to inject thrombin into the aneurysm.

The invention also relates to a method of manufacturing a endoprosthesis, in particular an endoprosthesis as described herein.

The method comprises the steps of:
Providing a base body of an endoprosthesis
Attaching a fiber at a first anchor point
Wrapping said fiber around the longitudinal axis of the endoprosthesis such that it extends at least around a full circumference of the endoprosthesis
Attaching the fiber at a second anchor point
Providing a fixing mechanism, preferably a fixing mechanism comprising a suture or a fabric strip, and attaching it along the longitudinal axis of the endoprosthesis such that the fixing mechanism fixes the fiber to the endoprosthesis at at least one attachment point
Cutting of the fiber at a cutting point, such that two ends of the fiber can extend away from the fixing mechanism.

In an alternative embodiment, the method comprises the steps of:
Providing a base body for an endoprosthesis
Determining at least one anchor point according to a predetermined geometry
Attaching a fiber to the at least one anchor point.

Preferably, each fiber is individually attached to a predefined anchor point by an automated process.

Preferably, the attachment is made by inserting an end of a fiber in a polymer layer of the endoprosthesis.

It will be understood by the person skilled in the art that all features described herein can of course be used alone or in any combination.

In the following, the invention is described in detail with reference to the following figures, showing:

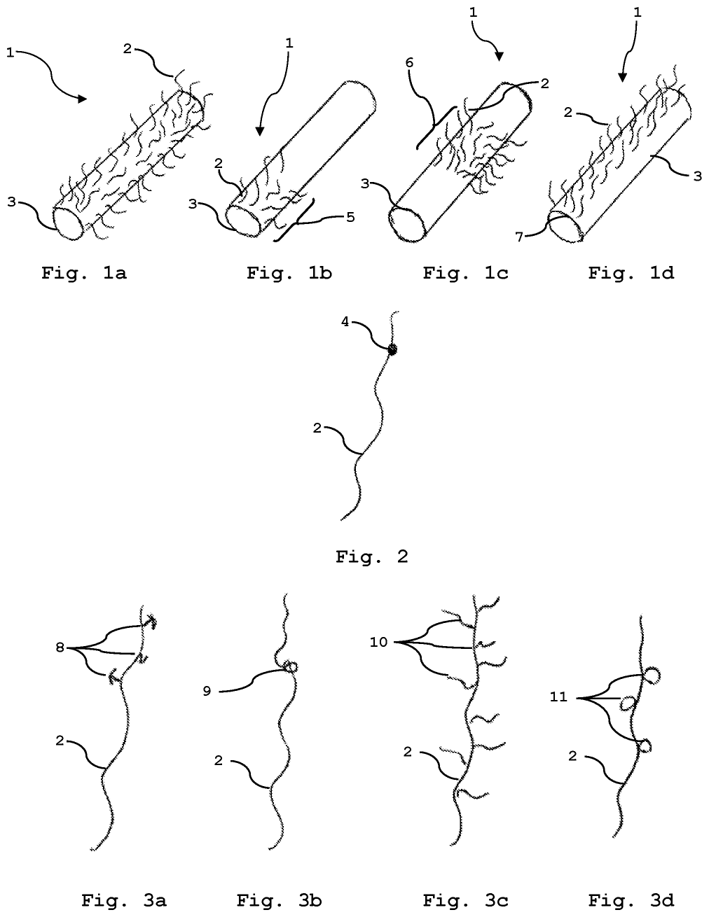

FIG. 1a-1d: different embodiments of an endoprosthesis.

FIG. 2: a fiber comprising a biosensor.

FIG. 3a-3d: different types of fibers.

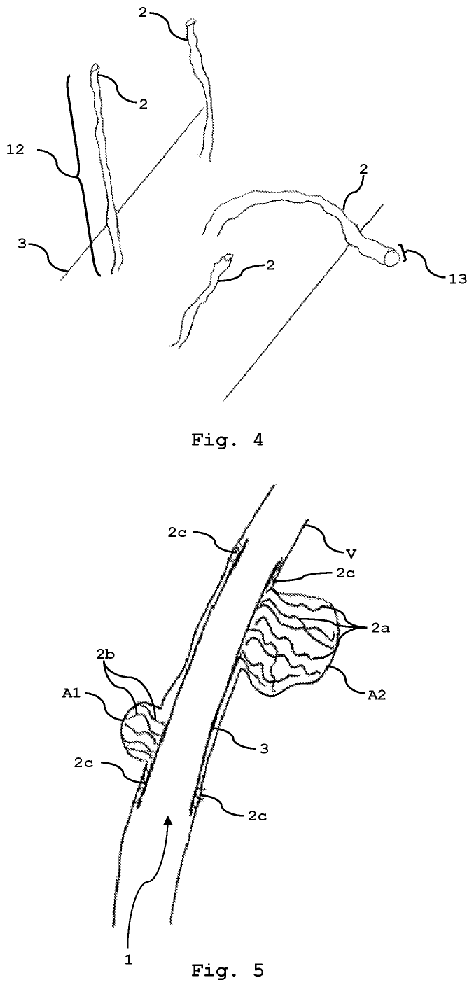

FIG. 4: a detailed depiction of fibers on a surface.

FIG. 5: a schematic depiction of an implanted endoprosthesis with two aneurysms.

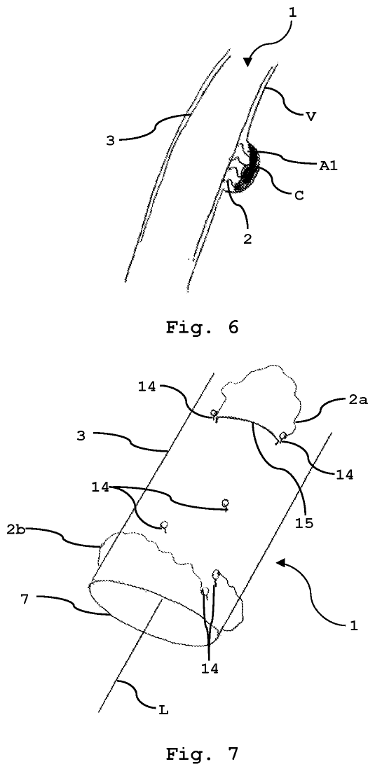

FIG. 6: a schematic depiction of an implanted endoprosthesis with an aneurysm.

FIG. 7: an alternative embodiment of an endoprosthesis.

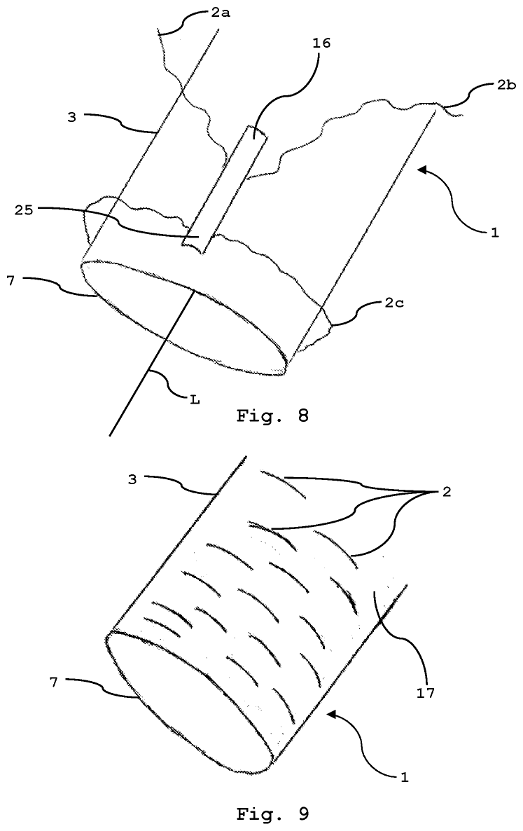

FIG. 8: another alternative embodiment of an endoprosthesis.

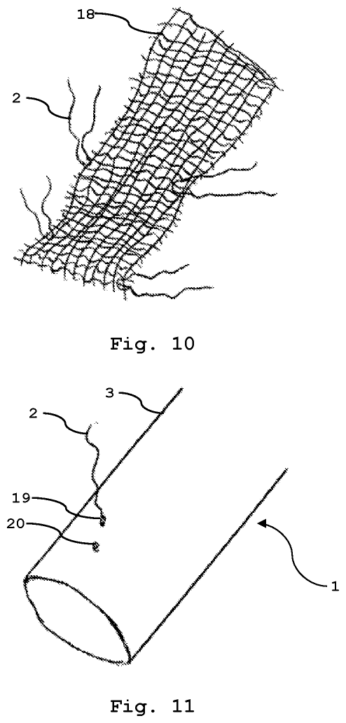

FIG. 9: another alternative embodiment of an endoprosthesis.

FIG. 10: a schematic depiction of a fabric with fibers.

FIG. 11: another alternative embodiment of an endoprosthesis.

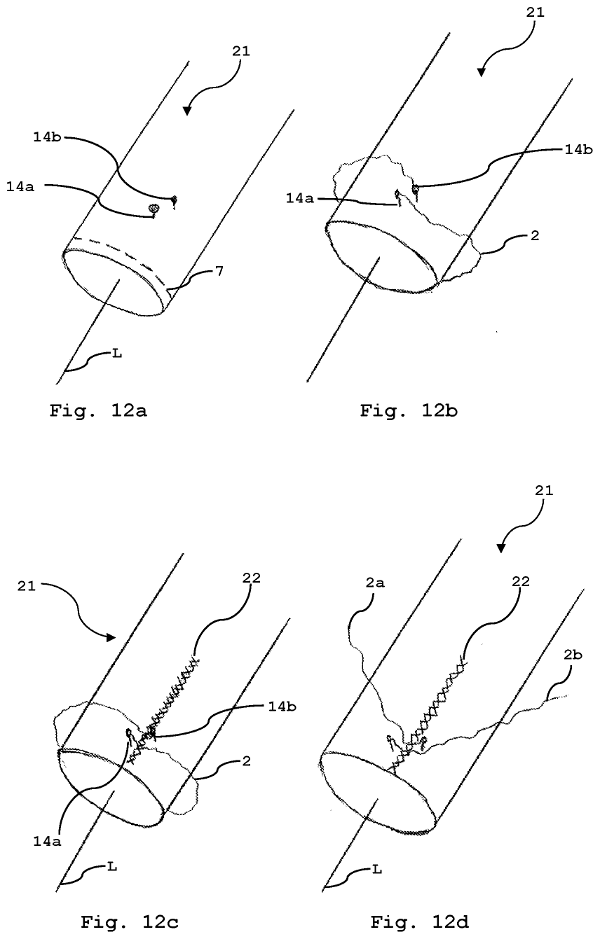

FIG. 12a-12d: a schematic illustration of a method to manufacture an endoprosthesis

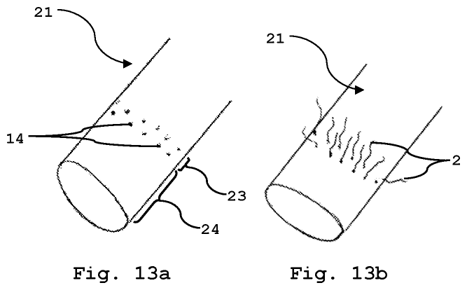

FIG. 13a-13b: a schematic illustration of an alternative method to manufacture an endoprosthesis.

FIG. 1 shows schematically a particularly preferred embodiment of an endoprosthesis 1. The outer surface of a body 3 of the endoprosthesis is entirely provided with thrombogenic fibers 2. Here the fibers are spread evenly over the entire surface. By contrast, FIGS. 1b-1d show different embodiments of an endoprosthesis 1 wherein only a part of the body 3 is provided with thrombogenic fibers 2. In FIG. 1b, only a distal part 5 of the body 3 of the endoprosthesis body 3 provided with fibers 2. Of course, it would also be possible to only provide the endoprosthesis body 3 with fibers on a proximal end instead, or on both ends. FIG. 1c shows an embodiment wherein only a middle part 6 of the endoprosthesis body 3 is provided with fibers 2. Finally, FIG. 1d shows an embodiment of the endoprosthesis 1 where only a part, for example half of the circumference 7, is provided with fibers 2. However, there is no gradient in the density of fibers along the longitudinal axis. On the part of the circumference 7 that is provided with fibers 2, the fibers are spread evenly from the distal to the proximal end of the endoprosthesis. On the other part of the circumference 7, there are no fibers at all. It shall be understood any of the embodiments described in FIGS. 1a-1d can be combined with any of the features or embodiments described below.

FIG. 2 shows a fiber 2 that is provided with a biomarker 4. The biomarker comprises molecules than can attach to other molecule that can indicate an inflammation. When attached to such molecules, the biomarker 4 changes its optical properties such that an inflammation can be detected easily.

FIG. 3a-3d show, by way of example, different types of surface textures of thrombogenic fibers. FIG. 3a shows an embodiment wherein micro-hooks 8 are spread over the surface of the fiber 2. The micro-hooks 8 consist, in this example, of metallic anchor-shaped and pointed pieces that can mechanically engage in different types of tissue. Micro-hooks are thus particularly advantageous in applications where the fibers need attach to different types of surfaces simultaneously, or if the exact nature of the surface is not known before the treatment. For example, the metallic micro-hooks 8 shown here may help attach the fibers to a blood clot in an aneurysm, while other micro-hooks may provide attachment to a vessel wall. Of course, it would also be possible to use micro-hooks of other biocompatible materials such as polymers.

FIG. 3b shows a fiber 2 comprising a knot 9. Such a knot 9 provides better retention from a coagulum that forms around the fiber and is particularly easy and cheap to manufacture because no additional material is needed. Instead, the knot can be formed from the fiber itself.

FIG. 3c shows an embodiment of a fiber 2 wherein secondary fibers 10 extend from the fiber 2. The mechanism of increased retention at a blood coagulum is the same as for a fiber containing a knot 9 as shown in FIG. 3b. However, secondary fibers as shown here provide the additional advantage that different lengths or types of secondary fibers 10 can be used along the length of the fiber 2. Thus, the attachment strength as well as the thrombogenicity can be tuned with more versatility.

FIG. 3d show an embodiment of a fiber 2 comprising loops 11. It will be understood by a person skilled in the art the textures shown here are mere examples.

FIG. 4 shows a close-up schematic view of the surface of an endoprosthesis body 3. Several fibers 2 are arranged on the surface. The fibers 2 are individually adapted in the diameter 13 and length 12 to the patient that is treated in this situation. It is well visible that here, the fibers have a varying diameter 13 along their length. The variation is random and the increased surface-to-volume ratio further promotes blood coagulation. It would of course be possible to adapt the fibers 2 to have any diameter profile. For example, the fibers could also become thicker toward their free end, or thinner, or have an hour-glass shape. Here, the fibers are made of biodegradable polylactic acid are adapted such that they degrade in the human body within eight months.

FIG. 5 shows an example of a treatment with an endoprosthesis 1 according to the invention. In this illustration, the endoprosthesis 1 is in its deployed state in a human vessel V. The vessel V has two aneurysms A1, A2 of different sizes. The embodiment of the endoprosthesis 1 shown here is particularly adapted to the treatment situation and the patient. The fibers 2 are arranged on the surface of the endoprosthesis body 3 at the location of the aneurysms A1, A2. At the location of the larger aneurysm A2, the fibers 2a are longer to fill substantially the entire volume of the aneurysm. At the location of the smaller aneurysm A1, the fibers 2b are shorter but achieve the same effect because of the smaller aneurysm size. In addition, short fibers 2c are arranged around the entire circumference of the endoprosthesis 2 at the proximal and the distal end in order to prevent endoleaks.

FIG. 6 shows another example of a treatment of an aneurysm A1 with an endoprosthesis 1 according to the invention. The vessel V in this example has only one aneurysm A1. The embodiment shown here is only provided with fibers 2 at the location of the aneurysm A1, while the rest of the surface of the endoprosthesis body 3 is free of fibers. The fibers 2 here are adapted in their length to fill the entire volume of the aneurysm A1 and additionally are provided with an active agent that additionally promotes blood coagulation. Thus, the formation of a blood clot C is relatively rapid. In addition, the fibers are provided with micro-hooks (not shown) on their free ends that significantly increase the retention of the fibers in the blood clot C.

FIG. 7 shows another embodiment of an endoprosthesis 1. The surface of the endoprosthesis body 3 is provided with several anchor points 14 to which fibers 2 are attachable. One fiber 2a is attached to two anchor points in such a way as to form a loop. This is achieved by employing a fiber that has a length which is longer that the shortest distance 15 between the two anchor points to which is attached. Thus, the fiber 2a extends from the surface of the endoprosthesis body 3 and promotes blood coagulation and retention of the endoprosthesis 1. Another fiber 2b is arranged around the circumference 7 of the endoprosthesis body 3. Here, it extends exactly once around the longitudinal axis L of the endoprosthesis. Thus, the ends of the fiber 2a and the anchor points 14 to which they are attached are at the same angular position relative to the longitudinal axis L of the endoprosthesis. The anchor points shown here consist of loops. The ends of the fibers 2 have foots, here in the form of mechanical retainers, that can engage the loops and thus attach the fiber to the anchor points 14 and the endoprosthesis body 3. It shall be understood that the both arrangement of fibers depicted in FIG. 7, either as a partial loop extending away from the endoprosthesis 2a or as a loop 2b around the longitudinal axis L of the endoprosthesis, can be used individually or in combination.

FIG. 8 shows a more schematic illustration of an embodiment of an endoprosthesis 1. Here, the fibers are attached to the endoprosthesis body 3 the same way as the fiber 2b in FIG. 7. Briefly, the fibers 2 are primarily attached to an anchor point (not shown) and wrapped around the circumference 7 of the endoprosthesis body 3 before attaching to a second anchor point. Here, the anchor points are aligned along the longitudinal axis L of the endoprosthesis 1. For permanent attachment to the endoprosthesis, a fabric strip 16 made of Dacron is glued along the longitudinal axis L such that it covers the anchor point and thereby attaches the fibers 2a, 2b, 2c to the endoprosthesis body 3 at an attachment point 25. It will of course be understood by the person skilled in the art the strip 16 could be made of any other biocompatible material as well. In addition, while gluing of the strip is a preferred method of attaching the strip, other methods such as sewing, stitching, or stapling are also possible. The fiber 2c forms a loop around the longitudinal axis of the endoprosthesis and thus promotes blood coagulation all around the circumference. This is particularly advantageous to prevent endoleaks. The other two fibers 2a, 2b shown here initially consisted of only one fiber that was arranged in the same way as the fiber 2c. However, the fiber was cut at a cutting point (not shown) on the opposite side of the endoprosthesis body 3 such that two fibers 2a, 2b formed whose ends are free and extend away from the endoprosthesis. For example, this be done at the location of an aneurysm. It shall be understood that the both arrangement of fibers depicted in FIG. 7, either cut fibers 2a, 2b extending away from the endoprosthesis or formed as a loop 2c around the longitudinal axis L of the endoprosthesis, can be used individually or in combination.

FIG. 9 shows an embodiment of an endoprosthesis before deployment. Here, the fibers 2 are attached to the endoprosthesis body 3 along their length by means of a biodegradable glue 17, here a polylactide. Thus, all the fibers are contiguous with the surface of the endoprosthesis body 3 before deployment. Here, they are oriented along the circumference 7 of the endoprosthesis body 3. Because the glue 17 used here is biodegradable, the fibers will be released upon implantation and eventually extend away from the endoprosthesis body 3. Here, the glue is adapted to degrade in the human body within five months. However, the degradation rate can be tuned to any appropriate depending on the patient and the application.

FIG. 10 shows schematically a fabric 18 that can be used to manufacture an endoprosthesis according to the invention. The fabric 18 is woven and has fibers 2 that are directly integrated into the fabric 18, here by weaving fibers during manufacturing of the fabric 18.

FIG. 11 shows schematically an attachment of fibers via a connection interface 20. The endoprosthesis body 3 comprises a magnetic connection interface. The fiber 2 has a foot which is a magnetic as well. Thus, the fiber is automatically attracted to the connection interface 20 on the endoprosthesis body 3 and attaches there. This method is particularly advantageous because the fibers 2 are automatically pulled to the desired location by magnetic forces. Thus, it provides for a simple mechanism to accurately distribute the fibers 2 on the endoprosthesis body 3.

FIGS. 12a-12d show schematically a method of manufacturing an endoprosthesis. A base body 21 of an endoprosthesis is provided as shown in FIG. 12a. It has a longitudinal axis L and a circumference 7. Two anchor points 14a, 14b are provided on the surface of the base body 21. Here, the anchor points are not at the same angular position relative to the longitudinal axis L, meaning that a hypothetical straight line connecting them is not parallel to the longitudinal axis L. As shown in FIG. 12b, a fiber 2 is attached to one of the anchor points 14a. The fiber 2 is subsequently wrapped around the base body 21 and its longitudinal axis L and attached to the anchor point 14b. Because the two anchor points 14a, 14b are not at the same angular position, the fiber 2 extends over more than one circumference in this example. Here, this is particularly advantageous because it allows for an arrangement of a suture 22 as in between the anchor points 14a, 14b. The suture 22 is provided on the surface of the base body 21 and arranged along the longitudinal axis L. It is placed such that is fixes the fiber 2 in between the two anchor points 14a, 14b. Finally, the fiber 2 is cut on a side opposite of the suture 22, leading to the endoprosthesis shown in FIG. 12d. The cut had divided the fiber into two segments 2a, 2b, each extending away from the endoprosthesis base body 21.

FIGS. 13a-13b show an alternative method to manufacture an endoprosthesis. An endoprosthesis base body 21 is provided and the location of anchor points is determined based on a planned treatment. Here, the predetermined geometry consists of a band 23 around the circumference of the base body 21. This is particularly advantageous to prevent endoleaks because the formation of blood coagula around the circumference of the endoprosthesis can seal the vessel. In addition, here, a safety distance 24 is kept from the end of the endoprosthesis to further prevent the formation of blood clots that could be rinsed off into the blood stream. In a next step, illustrated in FIG. 13b, fibers 2 are attached to the anchor points 14, thus forming an area on the endoprosthesis surface provided with fibers according to a predetermined geometry.

1.-37. (canceled)

38. An endoprosthesis, comprising at least one body part, wherein at least one area of an outer surface of the at least one body part is provided with thrombogenic fibers.

39. The endoprosthesis according to claim 38, wherein the thrombogenic fibers are one of biodegradable and adapted to elute a drug.

40. The endoprosthesis according to claim 38, wherein at least one of the thrombogenic fibers comprises a biomarker or a biosensor.

41. The endoprosthesis according to claim 38, wherein the thrombogenic fibers are made of an elastic material and are adapted to expand upon deployment of the endoprosthesis.

42. The endoprosthesis according to claim 38, wherein at least one of the length, diameter, and density of the thrombogenic fibers is adapted to minimize or prevent endoleaks.

43. The endoprosthesis according to claim 38, wherein at least one of the length, diameter, and density of the thrombogenic fibers is optimized based on the characteristics of the aneurism to be treated.

44. The endoprosthesis according to claim 38, wherein the thrombogenic fibers comprise an active agent.

45. The endoprosthesis according to claim 44, wherein the active agent inhibits at least one of plasmin and metallo protease.

46. The endoprosthesis according to claim 38, wherein the thrombogenic fibers comprise a pharmaceutical substance that promotes and/or increases coagulation.

47. The endoprosthesis according to claim 38, wherein the thrombogenic fibers comprise two ends, both of which are attached or attachable to the endoprosthesis by way of two separate anchor points.

48. The endoprosthesis according to claim 47, wherein the shortest distance between the anchor points along the surface of the endoprosthesis in its deployed form is shorter than the length of the fiber that is connected to the two anchor points.

49. The endoprosthesis according to claim 38, wherein at least one of the thrombogenic fiber extends around the longitudinal axis of the endoprosthesis at least once.

50. The endoprosthesis according to claim 49, comprising a strip or suture extending substantially along the longitudinal axis, wherein said strip or suture is attached or attachable to the at least one thrombogenic fiber at least one attachment point.

51. The endoprosthesis according to claim 50, wherein the at least one thrombogenic fiber that is oriented along a circumference is cuttable or cut at at least one cutting point so that cut ends of the fibers extend away from the attachment point of said strip or suture.

52. The endoprosthesis according to claim 38, wherein the thrombogenic fibers are attached to the endoprosthesis along their length by means of an adhesive composition and wherein the adhesive composition is biodegradable so that release of the fiber from the surface of the endoprosthesis occurs only post-implantation due to degradation of the adhesive composition.

53. The endoprosthesis according to claim 38, wherein a diameter of at least one of the thrombogenic fibers, is varying from one end of the fiber towards another end of the fiber over at least a section of the fiber.

54. The endoprosthesis according to claim 38, comprising a fabric with directly integrated thrombogenic fibers.

55. The endoprosthesis according to claim 54, wherein the fabric is woven, braided, or knitted.

56. The endoprosthesis according to claim 38, wherein the thrombogenic fibers comprise a foot that promotes attachment into a endoprosthesis layer.

57. The endoprosthesis according claim 38, comprising at least two different types of thrombogenic fibers.

58. The endoprosthesis according to claim 53, wherein the at least two different types of thrombogenic fibers differ in at least one of length, diameter, or composition.

59. The endoprosthesis according to claim 38, comprising at least one nonthrombogenic fiber.

60. The endoprosthesis according claim 38, wherein at least a part of the endoprosthesis is manufactured by additive manufacturing.

61. The endoprosthesis according claim 53, wherein at least the fibers are manufactured by additive manufacturing.

62. The endoprosthesis according to claim 61, wherein the whole endoprosthesis is manufactured by additive manufacturing.

63. The endoprosthesis according to claim 38, wherein the thrombogenic fibers are provided with a connection interface for separately attaching the fibers to the endoprosthesis such as to allow subsequent fixation of the fibers to the endoprosthesis.

64. The endoprosthesis according to claim 63, wherein the connection interface is adapted to establish a connection based on magnetic forces.

65. The endoprosthesis according to claim 38, comprising an inner and an outer layer, wherein the thrombogenic fibers are attached or attachable to the outer layer, and the outer layer is attached or attachable to the inner layer.

66. The endoprosthesis according to claim 65, wherein the inner layer and the outer layer are attached or attachable by means of a glue and/or a suture.

67. A method of manufacturing an endoprosthesis, comprising the steps of:
providing a base body of an endoprosthesis;
attaching a fiber at a first anchor point;
wrapping said fiber around the longitudinal axis of the endoprosthesis such that it extends at least around a full circumference of the endoprosthesis;
attaching the fiber at a second anchor point;
providing a fixing mechanism, and attaching it along the longitudinal axis of the endoprosthesis such that the fixing mechanism fixes the fiber to the endoprosthesis at at least one attachment point; and
cutting the fiber at a cutting point, such that two ends of the fiber can extend away from the fixing mechanism.
